(19) United States
(12) Patent Application Publication
(10) Pub. No.: US 2025/0318029 A1
(43) Pub. Date: Oct. 9, 2025

(54) HIGH FIDELITY, LOW-BLUE LIGHT SYSTEM

(71) Applicant: KORRUS, INC., Los Angeles, CA (US)

(72) Inventors: Paul Kenneth Pickard, Los Angeles, CA (US); Raghuram L.V. Petluri, Los Angeles, CA (US); Sina Afshari, Los Angeles, CA (US)

(21) Appl. No.: 19/244,794

(22) Filed: Jun. 20, 2025

Related U.S. Application Data
(63) Continuation of application No. PCT/US23/85380, filed on Dec. 21, 2023.
(60) Provisional application No. 63/434,320, filed on Dec. 21, 2022.

(51) Int. Cl. H05B 45/20; H05B 47/155; H05B 47/17

(52) U.S. Cl. CPC H05B 45/20; H05B 47/155; H05B 47/17

(57) ABSTRACT

A lighting system for emitting emitted light, comprising: (a) multiple independently controlled channels, each channel representing a point on a chromaticity space diagram, said multiple channels comprising at least; (i) a blue channel having a blue point on said chromaticity space diagram; (ii) a cyan channel having a cyan point on said chromaticity space diagram; (iii) a red channel having a red point on said chromaticity space diagram; and (iv) a violet-pumped low-blue channel having a low-blue point on said chromaticity space diagram, wherein said low-blue point is within a 7-step MacAdam ellipse of the Planckian locus; and (b) a controller for independently controlling each of said multiple channels to vary said emitted light.

### CROSS REFERENCE TO RELATED APPLICATIONS

The present patent application is a continuation of International Application No. PCT/US23/85380, filed Dec. 21, 2023, which claims the benefit of U.S. Provisional Patent Application 63/434,320, filed Dec. 21, 2022, the entire disclosures of each of which are hereby incorporated by reference.

### FIELD OF INVENTION

The present invention relates, generally, to high fidelity lighting, and, more specifically, to high fidelity lighting having a mode to minimize circadian stimulation.

### BACKGROUND OF INVENTION

A wide variety of light emitting devices are known in the art including, for example, incandescent light bulbs, fluorescent lights, and semiconductor light emitting devices such as light emitting diodes (“LEDs”). Of particular interest herein, are LEDs having a high quality of light.

The quality of light may be described in different ways. For example, one commonly used resource is 1931 CIE (Commission Internationale de l'Éclairage) Chromaticity Diagram. The 1931 CIE Chromaticity Diagram maps out the human color perception in terms of two CIE parameters x and y. The spectral colors are distributed around the edge of the outlined space, which includes all of the hues perceived by the human eye. The boundary line represents maximum saturation for the spectral colors, and the interior portion represents less saturated colors including white light. The diagram also depicts the Planckian locus, also referred to as the black body locus (BBL), with correlated color temperatures, which represents the chromaticity coordinates (i.e., color points) that correspond to radiation from a black-body at different temperatures. Illuminants that produce light on or near the BBL can thus be described in terms of their correlated color temperatures (CCT). These illuminants yield pleasing “white light” to human observers, with general illumination typically utilizing CCT values between 1,800 K and 10,000 K.

Color rendering index (CRI) is described as an indication of the vibrancy of the color of light being produced by a light source. In practical terms, the CRI is a relative measure of the shift in surface color of an object when lit by a particular lamp as compared to a reference light source, typically either a black-body radiator or the daylight spectrum. The higher the CRI value for a particular light source, the better that the light source renders the colors of various objects it is used to illuminate.

Color rendering performance may be characterized via standard metrics known in the art. Fidelity Index (Rf) and the Gamut Index (Rg) can be calculated based on the color rendition of a light source for 99 color evaluation samples (“CES”). The 99 CES provide uniform color space coverage, are intended to be spectral sensitivity neutral, and provide color samples that correspond to a variety of real objects. Rf values range from 0 to 100 and indicate the fidelity with which a light source renders colors as compared with a reference illuminant. In practical terms, the Rf is a relative measure of the shift in surface color of an object when lit by a particular lamp as compared to a reference light source, typically either a black-body radiator or the daylight spectrum. The higher the Rf value for a particular light source, the better that the light source renders the colors of various objects it is used to illuminate. The Gamut Index Rg evaluates how well a light source saturates or desaturates the 99 CES compared to the reference source.

Applicant previously disclosed a revolutionary LED lighting system that provides on-Planckian white light having a very high Rf over a wide CCT range—e.g., from 1800 K to 10000 K. See, e.g., PCT/US2018/020787. In one embodiment, the system has a blue channel, a red channel, a short-blue cyan channel (green) and a long-blue cyan channel (cyan). Although this system provides unsurpassed light quality and tunability in an LED light system, Applicant recognizes the need to reduce circadian stimulation (CS). Specifically, due to the long-blue cyan channel, the emitted light ranges from normal to high CS, and thus may result in unwanted CS.

More specifically, blue light inhibits melatonin production and may negatively affect the human body's natural sleep cycle. As used herein, the term “circadian-stimulating energy characteristics” refers to any characteristics of a spectral power distribution that may have biological effects on a subject. Circadian-stimulating energy characteristics may be described in various terms, including, for example, circadian-stimulating energy (CSE), circadian stimulation (CS), Equivalent Melanopic Lux (EML), and M/P ratio. Of particular interest herein are EML and M/P ratio. EML provides a measure of photoreceptive input to circadian and neurophysiological light responses in humans. The M/P ratio compares the melanopic (ipRGC) potential to the light source's ability to produce light for daytime detail vision (photopic vision).

Applicant recognizes the need to provide LED lamps that can provide high-quality, white light across a range of CCT values while simultaneously moderating CS. The present invention fulfills this need, among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

As mentioned above, the light system disclosed in PCT/US2018/020787 is a revolutionary LED lighting system that provides on-Planckian white light having a very high Rf over a wide temperature range—e.g., from 1800 K to 10000 K. In one embodiment, the system has a blue channel, a red channel, a short-blue cyan channel (green) and a long-blue cyan channel (cyan). Although this system provides unsurpassed light quality and tunability for an LED light system, Applicant recognizes the need to reduce EML. Specifically, due to the long-blue cyan channel, the light ranges from typical EML (for off the shelf LEDs) to high EML, and thus may result in unwanted circadian stimulation.

Accordingly, Applicant discloses herein introducing a low-blue channel. More specifically, in one embodiment, Applicant substitutes the high EML short-blue cyan channel for a violet-pumped low-blue channel. The combination of the long-blue cyan (cyan) channel with the low-blue channel provides a low-blue alternative to the short-blue cyan (green) channel. Such a light system offers various modes, including a high circadian stimulation modes (using long-blue cyan channel), a low circadian stimulation (using the low-blue channel), and a high fidelity mode (using both the long-blue cyan and low-blue channels), without sacrificing light quality. More specifically, Applicant expected a loss of Rf in high fidelity mode because the light from the low-blue channel—which has relatively low Rf—needs to fill in the spectrum that short blue cyan filled previously. But that did not happen. Rather, Applicant discovered unexpectedly that the high-fidelity mode is almost as good with the low-blue channel as it was with short-blue Cyan channel.

In one particular embodiment, the low-blue channel is near or on the Planckian locus, such that, when in a low EML mode, the emitted light from the red, low-blue and blue channels is near the Planckian locus, and requires just a small contribution, if any, from the cyan channel to pull the light up to or slightly over the Planckian locus. Although Applicant expected the Rf values to drop in the emitted light because of the reduction in light in the range of 440-490, unexpectedly, the resulting Rf values are very good. Therefore, among other things, the invention involves the unexpected robust contribution of the low-blue channel to the high fidelity mode, thereby minimizing the drop in Rf.

In one embodiment, the invention relates to a lighting system for emitting emitted light, said system comprising: (a) multiple independently controlled channels, each channel representing a point on a chromaticity space diagram, said multiple channels comprising at least; (i) a blue channel having a blue point on said chromaticity space diagram; (ii) a cyan channel having a cyan point on said chromaticity space diagram; (iii) a red channel having a red point on said chromaticity space diagram; and (iv) a violet-pumped low-blue channel having a low-blue point on said chromaticity space diagram, wherein said low-blue point is within a 7-step MacAdam ellipse of the Planckian locus; and (b) a controller for independently controlling each of said multiple channels to vary said emitted light.

### BRIEF DESCRIPTION OF FIGURES

FIG. 1 shows one embodiment of the lighting system of the present invention.

FIG. 2A shows the spectral power distribution (SPD) for each channel of one embodiment of the lighting system of the present invention.

FIG. 2B shows a chromaticity diagram of one embodiment of the lighting system of the present invention.

FIG. 3A shows the Rf values across a wide CCT range for three different modes of one embodiment of the lighting system of the present invention.

FIG. 3B shows the m/p ratios across a wide CCT range for three different modes of one embodiment of the lighting system of the present invention.

FIG. 4A is a chromaticity diagram showing a region of the blue channel of one embodiment of the lighting system of the present invention.

FIG. 4B is a chromaticity diagram showing regions of the cyan and red channels of one embodiment of the lighting system of the present invention.

FIG. 5 is a chromaticity diagram showing specific regions of the cyan and red channels of one embodiment of the lighting system of the present invention.

FIG. 6 is a chromaticity diagram showing other regions of the cyan and red channels of one embodiment of the lighting system of the present invention.

FIG. 7 is a chromaticity diagram showing specific regions of the blue and red channels of one embodiment of the lighting system of the present invention.

FIG. 8 is a chromaticity diagram showing specific regions of the blue and red channels of one embodiment of the lighting system of the present invention.

FIG. 9 is a chromaticity diagram showing specific regions of the red channel of one embodiment of the lighting system of the present invention.

FIG. 10 a chromaticity diagram showing specific regions of the blue channel of one embodiment of the lighting system of the present invention.

FIG. 11A is a chromaticity diagram showing a region of the low-blue channel of one embodiment of the lighting system of the present invention.

FIG. 11B is a chromaticity diagram showing a more specific region of the low-blue channel of one embodiment of the lighting system of the present invention.

### DETAILED DESCRIPTION

Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

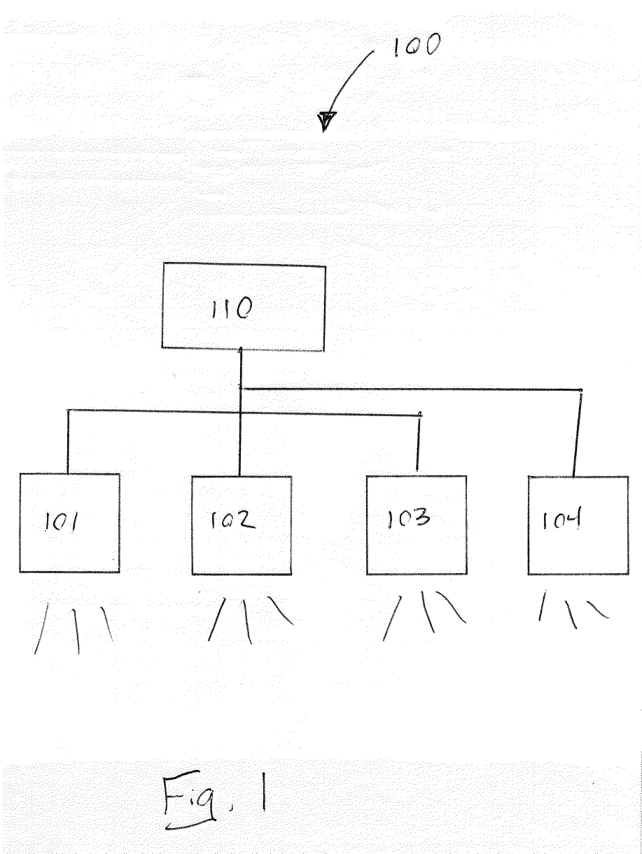

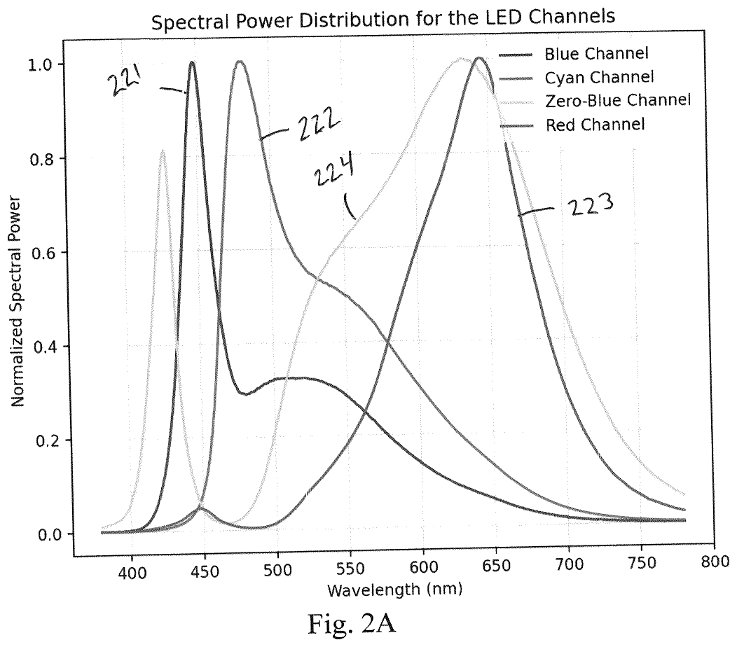

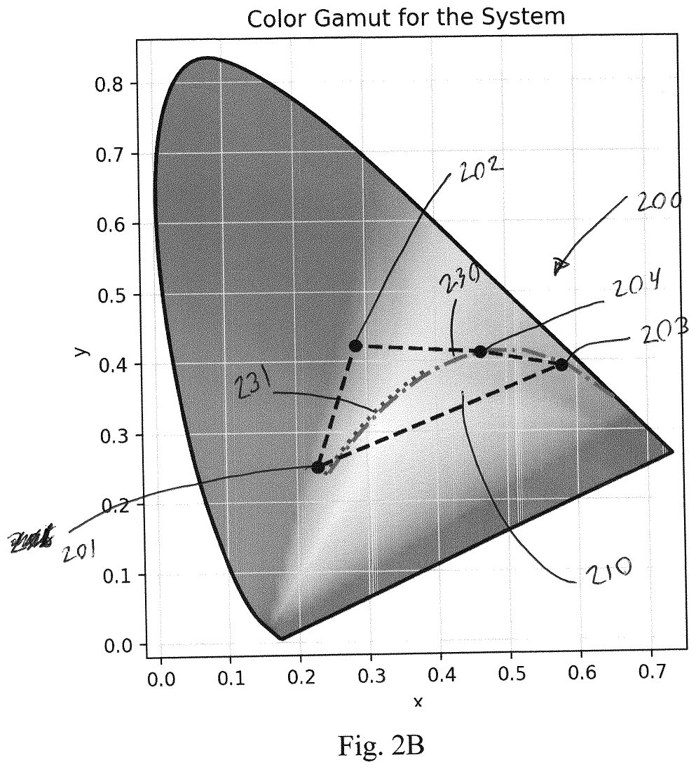

Referring to FIG. 1, one embodiment of the lighting system 100 of the present invention is shown. The system comprises multiple independently controlled channels, each channel representing a point on a chromaticity space diagram. The multiple channels comprises at least a blue channel 101 having a blue point 201 on said chromaticity space diagram 200 shown in FIG. 2B, a cyan channel 102 having a cyan point 202 on said chromaticity space diagram, a red channel 103 having a red point 203 on said chromaticity space diagram; and a violet-pumped low-blue channel 104 having a low-blue point 204 on said chromaticity space diagram, wherein said low-blue point is within a 7-step MacAdam of the BBL. The system also comprises a controller 110 for independently controlling each of said multiple channels to vary the emitted light being emitted from the lighting system.

### Modes

The configuration of these four channels allows the lighting system of the present invention to operate in at least three modes over a wide CCT range. Specifically, in one embodiment, the controller is configured to operate the lighting system to emit light in a low EML mode, a high EML mode, and a high fidelity mode. In the low EML mode, the blue, red, and low-blue channels are driven principally, with power to the cyan channel reduced or eliminated altogether. In the high EML mode, the blue, red, and cyan channels are driven principally, with power to the low-blue channel reduced or eliminated altogether. In the high fidelity mode, all four channels are powered across the CCT range to emit light with the highest RF values. In one embodiment, the controller powers different channels at different intensities in the different modes. Referring to Table 1, the relative power of each channel for each mode through the CCT range is given. In one embodiment, the relative contribution for each of said channel in each mode is within +/−20%, or within +/−10%, or within +/−5% of the values set forth in Table 1.

### High Fidelity Mode

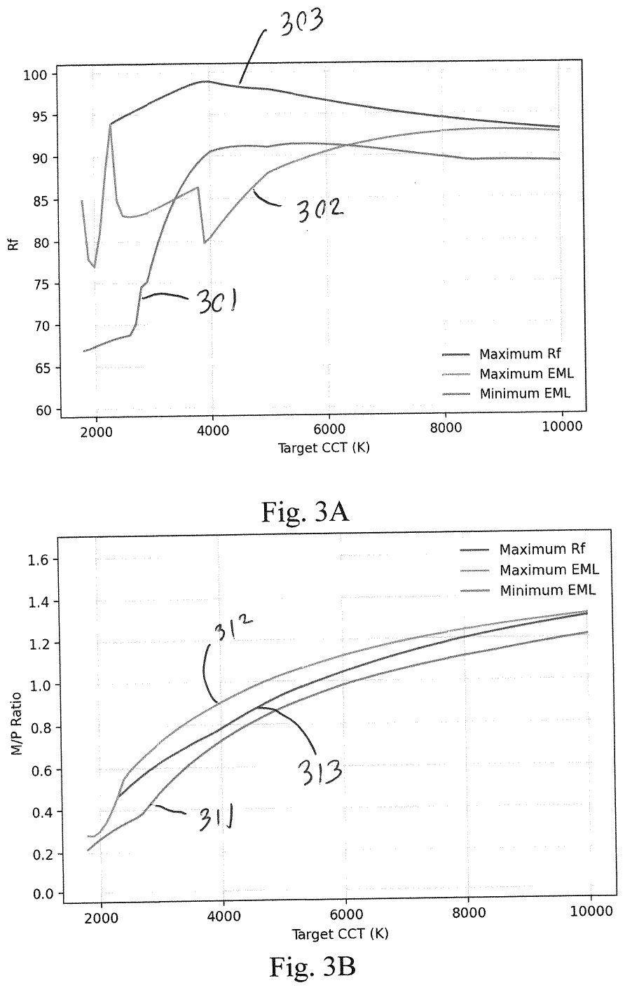

As mentioned above, Applicant discovered unexpectedly that the light system of the present invention is able to emit light having a fidelity (e.g., Rf) close to that of the high fidelity system disclosed in PCT/US2018/020787. For example, referring to FIG. 3A, the fidelity of the different modes of the embodiment of the system having channels described in FIG. 2A is shown. More specifically, this figure shows the RF values of the low EML mode 301, the high-EML mode 302, and the high fidelity mode 303 as a function of CCT. It is worthwhile to note that the high fidelity mode achieves RF values greater than 90 through a wide CCT range. In one embodiment, when in the high fidelity mode, the emitted light has an RF value of at least 90 between 2000 K and 80000 K or between 1800 K and 10000 K, or at least 95 between 2500 K and 7000 K, or at least 97 at 4000 K. Even the high-EML and the low EML modes have relatively high Rf values. For example, in one embodiment, the RF value for the emitted light regardless of the mode is at least 80 above 4000 k or between 4000 K and 10000 K, or at least 85 above 5000 k or between 5000 K and 10000 K.

### Low Eml Mode

As discussed above, an important aspect of the present invention is a light system having a low EML mode. In one embodiment, the low EML mode reduces blue light in the emitted light. In one embodiment, said emitted light has an overall SPD power and a blue light SPD power between 440 and 490 nm, wherein said blue SPD power is no greater than at least 5%, or 3%, or 2%, or 1% of said overall SPD power. Rather than blue light, in one embodiment, the present invention compensates for the blue light with violet light. In one embodiment, said emitted light has an overall SPD power and a violet SPD power between 380 and 420 nm, wherein said violet SPD power is at least 2%, or 3%, or 4%, or 5% of said overall SPD power.

The reduction in the blue component of light has a positive effect in reducing circadian stimulation. For example, referring to FIG. 3B, the circadian effects of the different modes of the embodiment of the system having channels described in FIG. 2A are shown. More specifically, the M/P ratios for the low EML mode 311, the high-EML mode 312, and a high fidelity mode 313 are shown. In the low EML mode 311, the M/P ratio is no greater than 1 below 6000 K, or no greater than 0.8 below 4000 K, or no greater than 0.6 below 3000 K.

### High EML MODE

In one embodiment, the lighting system of the present disclosure also has a high EML mode. Referring to back to FIG. 3B, in the high EML mode 311, the M/P ratio is no less than 0.8 above 4000 K, and no less than 1 above 5000 K.

### Channel Embodiments

Referring to FIG. 2A, the SPDs of each of the four channels of one embodiment of the present invention is shown. Specifically, the Fig. shows the SPD profiles for the blue channel 221, the cyan channel 222, the red channel 223, and the low-blue channel 224. In one embodiment, the SPD of each channel is within +/−20%, or within +/−10%, or within +/−5% of the SPD shown in FIG. 2A.

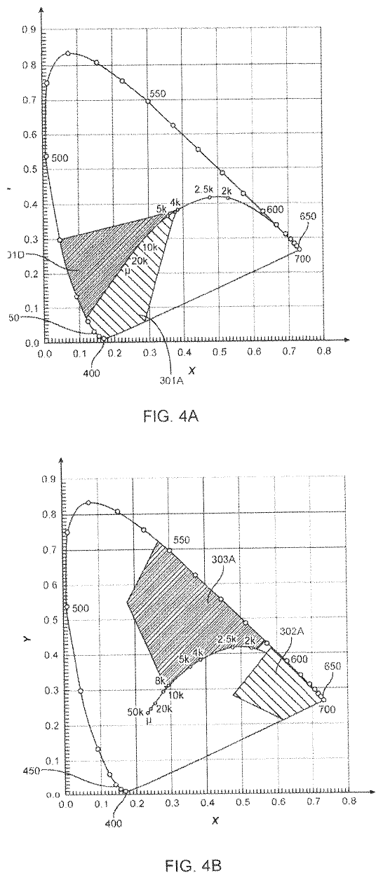

FIG. 2B shows a chromaticity diagram 200 for the four channel embodiment shown in FIG. 2A. Specifically, FIG. 4B shows gamut 210 of the four channel system defined by the blue point 201, the cyan point 202, the red point 203, and the low-blue point 204. It is worthwhile to note that essentially the entire BBL 230 is within the gamut 210 over a wide CCT range. The daylight spectrum locus 231 is also within the gamut 210.

### Low-Blue Channel

The low-blue channel is on or near the BBL and is pumped with a violet, ultraviolet, or near ultraviolet LED. The substitution of the low-blue channel for the short blue cyan channel of PCT/US2018/020787 represents a significant innovation, facilitating a low EML mode while still providing high fidelity in the high fidelity mode.

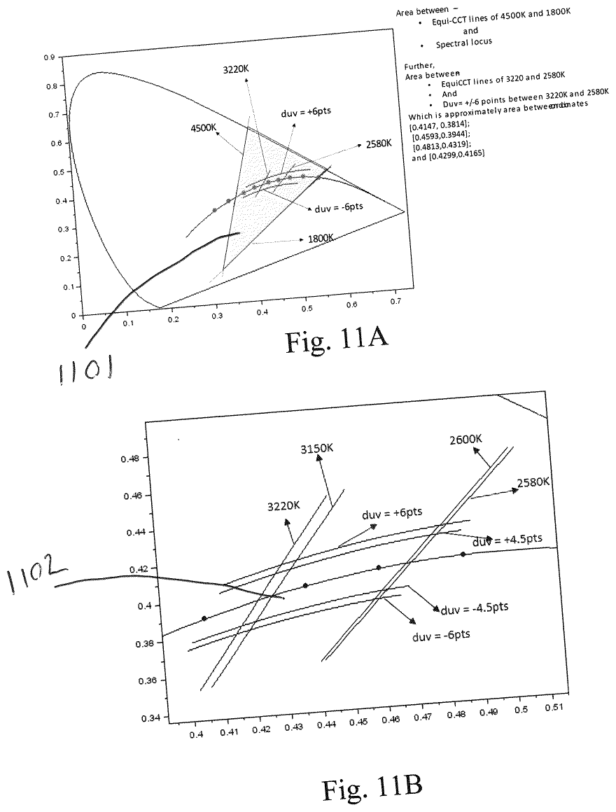

In one embodiment, the low-blue light point is within region 1101 as shown in the 1931 CIE Chromaticity Diagram of FIG. 11A. Specifically, region 1101 is defined by the equi-CCT lines of 1800 K and 4500 K and the spectral locus. In a more specific embodiment shown in FIG. 11B, region 1102 is defined by equi CCT lines of 3220 K and 2580 K, and Duv=+/−6 points between 3220 K and 2580 K. Alternatively, region 1102 may be defined by the area within the following ccx, ccy color coordinates [0.4147, 0.3814]; [0.4593, 0.3944]; [0.4813, 0.4319]; and [0.4299, 0.4165].

The low-blue channel has a relatively small blue portion. In one embodiment, the low-blue channel has a low-blue emitted light having an overall low-blue SPD power, and a blue SPD power between 440-490, wherein the blue SPD power is less than 2% of the overall low-blue SPD power. For example, in one embodiment, the blue SPD power is about 1.32% of the overall low-blue SPD power at 2700 K, or the blue SPD power is about 1.69% of the overall low-blue SPD power at 3000 K

As is known, in order to use LEDs to generate white light, one or more luminescent materials such as phosphors or quantum dots are used to convert some of the light emitted by one or more LEDs to light of one or more other colors. The combination of the light emitted by the LEDs that is not converted by the luminescent material(s) and the light of other colors that are emitted by the luminescent material(s) may produce a white or near-white light. In one embodiment, the LED is a violet pump LED. In one embodiment, the violet pump has a peak wavelength from 380 to 430 nm, or from 390 to 420 nm, or from 395 to 420 nm, or from 400 to 420 nm, or from 405 to 410 nm.

In one embodiment, the low-blue channel emits low-blue light which is no greater than a 7-step MacAdam ellipse from the BBL, or no greater than a 6-step MacAdam ellipse from the BBL, or no greater than a 5-step MacAdam ellipse from the BBL, or no greater than a 4-step MacAdam ellipse from the BBL, no greater than a 3-step MacAdam ellipse from the PPL.

### Blue Channel

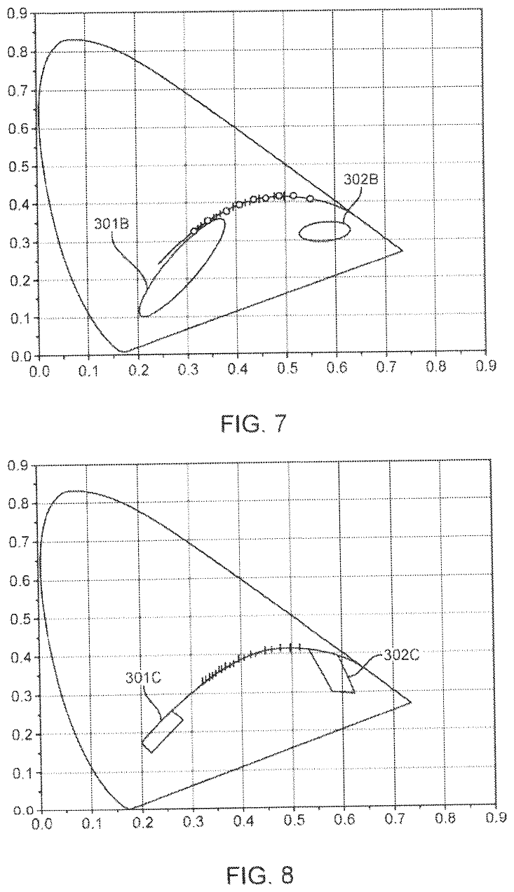

In some embodiments of the present disclosure, lighting systems can include blue channels that produce light with a blue color point that falls within a blue color range. In certain embodiments, suitable blue color ranges can include blue color ranges 301A-F. FIG. 4A depicts a blue color range 301A defined by a line connecting the cox, ccy color coordinates of the infinity point of the Planckian locus (0.242, 0.24) and (0.12, 0.068), the Planckian locus from 4000 K and infinite CCT, the constant CCT line of 4000 K, the line of purples, and the spectral locus. FIG. 4A also depicts a blue color range 301D defined by a line connecting (0.3806, 0.3768) and (0.0445, 0.3), the spectral locus between the monochromatic point of 490 nm and (0.12, 0.068), a line connecting the cex, ccy color coordinates of the infinity point of the Planckian locus (0.242, 0.24) and (0.12, 0.068), and the Planckian locus from 4000 K and infinite CCT. The blue color range may also be the combination of ranges 301A and 301D together. FIG. 7 depicts a blue color range 301B can be defined by a 60-step MacAdam ellipse at a CCT of 20000 K, 40 points below the Planckian locus. FIG. 8 depicts a blue color range 301C that is defined by a polygonal region on the 1931 CIE Chromaticity Diagram defined by the following cex, ccy color coordinates: (0.22, 0.14), (0.19, 0.17), (0.26, 0.26), (0.28, 0.23). FIG. 10 depicts blue color ranges 301E and 301F. Blue color range 301E is defined by lines connecting (0.231, 0.218), (0.265, 0.260), (0.2405, 0.305), and (0.207, 0.256).

### Cyan Channel

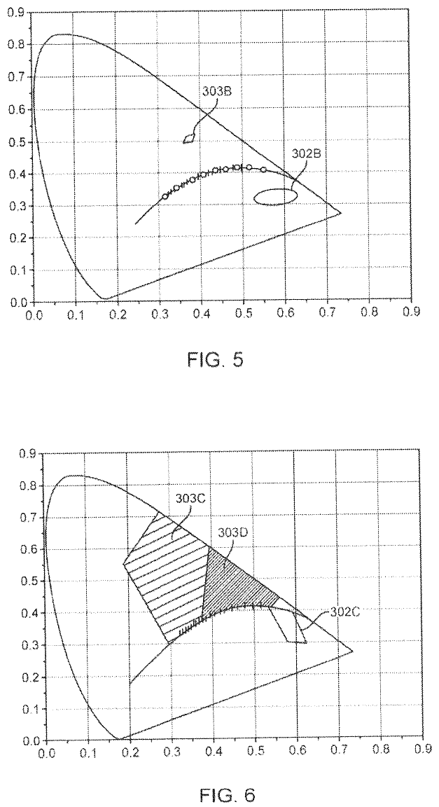

In some embodiments of the present disclosure, lighting systems can include long-blue-pumped cyan channels that produce light with a cyan color point that falls within a cyan color range. In certain embodiments, suitable cyan color ranges can include cyan color ranges 303A-E. FIG. 4B shows a cyan color range 303A defined by a line connecting the ccx, ccy color coordinates (0.18, 0.55) and (0.27, 0.72), the constant CCT line of 9000 K, the Planckian locus between 9000 K and 1800 K, the constant CCT line of 1800 K, and the spectral locus. FIG. 5 depicts some suitable color ranges for some embodiments of the disclosure. A cyan color range 303B can be defined by the region bounded by lines connecting (0.360, 0.495), (0.371, 0.518), (0.388, 0.522), and (0.377, 0.499). FIG. 6 depicts some further color ranges suitable for some embodiments of the disclosure. A cyan color range 303C is defined by a line connecting the ccx, ccy color coordinates (0.18, 0.55) and (0.27, 0.72), the constant CCT line of 9000 K, the Planckian locus between 9000 K and 4600 K, the constant CCT line of 4600 K, and the spectral locus. A cyan color range 303D is defined by the constant CCT line of 4600 K, the spectral locus, the constant CCT line of 1800 K, and the Planckian locus between 4600 K and 1800 K. In some embodiments, the long-blue-pumped cyan channel can provide a color point within a cyan color region 303E defined by lines connecting (0.497, 0.469), (0.508, 0.484), (0.524, 0.472), and (0.513, 0.459).

### Red Channel

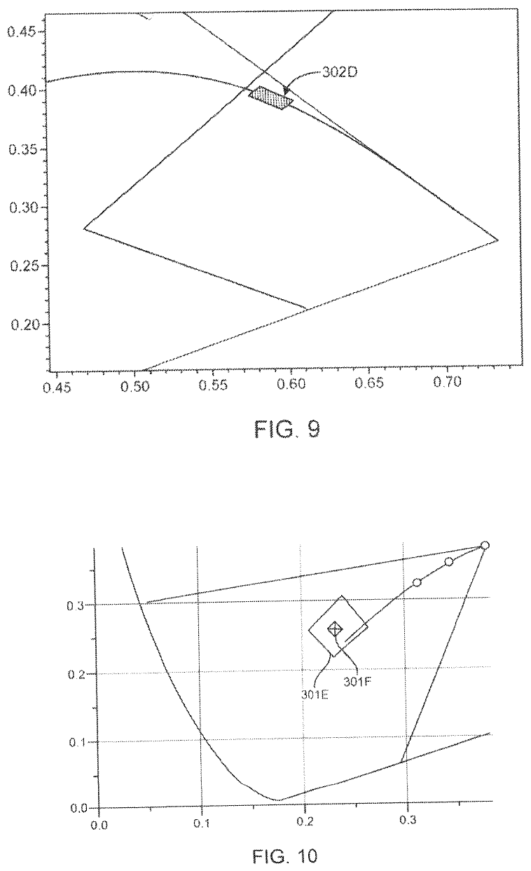

In some embodiments of the present disclosure, lighting systems can include red channels that produce light with a red color point that falls within a red color range. In certain embodiments, suitable red color ranges can include red color ranges 302A-D. FIG. 4B depicts a red color range 302A defined by the spectral locus between the constant CCT line of 1600 K and the line of purples, the line of purples, a line connecting the ccx, ccy color coordinates (0.61, 0.21) and (0.47, 0.28), and the constant CCT line of 1600 K. Red color ranges disclosed in the Long Red region are shown in FIG. 4B 302A. FIG. 5 depicts some suitable color ranges for some embodiments of the disclosure. A red color range 302B can be defined by a 20-step MacAdam ellipse at a CCT of 1200 K, 20 points below the Planckian locus. FIG. 6 depicts some further color ranges suitable for some embodiments of the disclosure. A red color range 302C is defined by a polygonal region on the 1931 CIE Chromaticity Diagram defined by the following ccx, ccy color coordinates: (0.53, 0.41), (0.59, 0.39), (0.63, 0.29), (0.58, 0.30). In FIG. 8, a red color range 302C is depicted and can be defined by a polygonal region on the 1931 CIE Chromaticity Diagram defined by the following ccx, ccy color coordinates: (0.53, 0.41), (0.59, 0.39), (0.63, 0.29), (0.58, 0.30). FIG. 9 depicts a red color range 302D defined by lines connecting the ccx, ccy coordinates (0.576, 0.393), (0.583, 0.400), (0.604, 0.387), and (0.597, 0.380).

Having thus described a few particular embodiments of the invention, various alterations, modifications, and improvements will readily occur to those skilled in the art. Such alterations, modifications, and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

What is claimed is:

1. A lighting system for emitting emitted light, said system comprising:
multiple independently controlled channels, each channel representing a point on a chromaticity space diagram, said multiple channels comprising at least;
a blue channel having a blue point on said chromaticity space diagram;
a cyan channel having a cyan point on said chromaticity space diagram;
a red channel having a red point on said chromaticity space diagram; and
a violet-pumped low-blue channel having a low-blue point on said chromaticity space diagram, wherein said low-blue point is within a 7-step MacAdam ellipse of the Planckian locus; and
a controller for independently controlling each of said multiple channels to vary said emitted light.

2. The system of claim 1, wherein low-blue light emitted from said low-blue channel is within a low-blue region on a 1931 CIE chromaticity diagram defined by the equi-CCT lines of 1800 K and 4500 K and the spectral locus.

3. The system of claim 2, wherein said low-blue region is defined by equi CCT lines of 3220 K and 2580 K, and Duv is +/−6 points between 3220 K and 2580 K.

4. The system of claim 2, wherein said low-blue region is defined by the area within the following ccx, ccy color coordinates [0.4147, 0.3814]; [0.4593, 0.3944]; [0.4813, 0.4319]; and [0.4299, 0.4165].

5. The system of claim 1, wherein blue light emitted from said blue channel is within regions 301A and 301D as shown in FIG. 4B.

6. The system of claim 1, wherein cyan light emitted from said cyan channel is within region 303A shown in FIG. 4B.

7. The system of claim 1, wherein red light emitted from said red channel is within region 302A shown in FIG. 4B.

8. The system of claim 1, wherein said controller is configured to control said multiple channels to emit said emitted light in at least one of a high fidelity mode, a low EML mode, or a high EML mode.

9. The system of claim 1, wherein the relative contribution for each of said channel in each mode is within +/−20%, or within +/−10%, or within +/−5% the values set forth in Table 1.

10. The system of claim 8, wherein said emitted light in said high fidelity mode has an Rf of at least 85, or at least 90, or at least 95.

11. The system of claim 8, wherein said emitted light in said low EML mode has an overall SPD power and a blue light SPD power between 440 and 490 nm, wherein said blue SPD power is no greater than at least 5%, or 3%, or 2%, or 1% of said overall SPD power.

12. The system of claim 8, wherein said emitted light in said low EML mode has an overall SPD power and a violet SPD power between 380 and 420 nm, wherein said violet SPD power is at least 2%, or 3%, or 4%, or 5% of said overall SPD power.

13. The system of claim 8, wherein said emitted light in said low EML mode has an M/P ratio no greater than 1 below 6000 K, or no greater than 0.8 below 4000 K, or no greater than 0.6 below 3000 K.

14. The system of claim 8, wherein said emitted light in said high EML mode has an M/P ratio no less than 0.8 above 4000 K, and no less than 1 above 5000 K.

15. The system of claim 8, wherein said emitted light in each of the modes has an Rf of at least 80.
